(19) United States
(12) Patent Application Publication
(10) Pub. No.: US 2016/0184011 A1
(43) Pub. Date: Jun. 30, 2016

(54) DUAL CATHETER ABLATION SYSTEM

(71) Applicant: Subramaniam C. KRISHNAN, Sacramento, CA (US)

(72) Inventor: Subramaniam C. KRISHNAN, Sacramento, CA (US)

(21) Appl. No.: 15/063,950

(22) Filed: Mar. 8, 2016

Related U.S. Application Data
(63) Continuation of application No. PCT/IB2014/064596, filed on Sep. 17, 2014.
(60) Provisional application No. 61/879,036, filed on Sep. 17, 2013.

(51) Int. Cl. A61B 18/14; A61M 25/10; A61B 18/04; A61B 18/02; A61B 18/24; A61B 18/18

(52) U.S. Cl. CPC A61B 18/1492; A61B 18/24; A61B 18/1815; A61B 18/04; A61B 18/02; A61B 18/18; A61M 25/10; A61B 2018/046; A61B 2018/048; A61B 2018/0212; A61B 2018/00577

(57) ABSTRACT

A dual catheter ablation system with an arterial catheter and a venous catheter and at least two magnetic elements, a first magnetic element placed in the arterial catheter and a second magnetic element placed in the venous catheter, where at least one of the arterial catheter or the venous catheter carries an ablating electrode. The first magnetic element has a predefined polarity and the second magnetic element has an opposite polarity with respect to the pre-defined polarity. A protective sheath is provided for enclosing the catheter pair, the first magnetic element and the second magnetic element. The dual catheter ablation system is configured to be placed inside an anatomical region such that a target tissue is in between the first magnetic element and the second magnetic element, bringing the ablating electrode in close proximity to a target tissue.

### CROSS-REFERENCE TO RELATED APPLICATIONS

This application is a continuation of PCT International Appl. No. PCT/IB2014/064596 filed Sep. 17, 2014, which claims benefit of priority to U.S. Provisional Patent Appl. No. 61/879,036, filed on Sep. 17, 2013, the content of each of which is incorporated herein by reference in its entirety.

### FIELD OF THE INVENTION AND USE OF INVENTION

The invention relates generally to medical devices and more specifically to a catheter system for percutaneous ablation of perivascular tissues, useful for cardiovascular procedures.

### PRIOR ART AND PROBLEM TO BE SOLVED

Renal sympathetic denervation (RSDN), also referred as renal denervation (RDN), is a minimally invasive, endovascular catheter based procedure using ablation for treating resistant hypertension (high blood pressure). In this procedure, radiofrequency (RF) or other energy pulses are applied to the wall of the renal arteries using a catheter, and the nerves in the vascular wall are denuded of nerve endings by the ablation caused by the energy pulses. This causes reduction of renal sympathetic afferent and efferent activity and blood pressure can be decreased. Renal sympathetic denervation has been shown to be effective in treating “difficult to control” or resistant hypertension.

Thus far, the most commonly used ablation technique and technology in such procedures has consisted of delivery of unipolar radiofrequency energy using an arterial endoluminal approach i.e. a catheter within the lumen of the renal artery is placed against the arterial wall and RF energy is delivered. With this approach, the nerve fibers and ganglia in direct contact with the arterial wall are more likely to be successfully ablated. Nerve structures further away are less likely to be affected, thus limiting the effectiveness of this approach. To ablate structures not in direct contact with the vessel wall, higher power will be needed and this could increase the likelihood of damaging the renal arterial wall, causing dissection, stenosis or thrombosis.

Typically, the catheter used for unipolar RF ablation is positioned adjacent to the abnormal or target tissue. High-frequency electrical energy is then passed between the ablation electrode and an indifferent electrode (ground electrode) that is generally a skin patch. The small area of target tissue under the tip of the ablation catheter is heated by this high-frequency energy, creating a lesion due to coagulation necrosis that then develops into a scar.

Prior experience in procedures of the heart has shown that attempts to ablate the entire thickness of the target tissue in the atrium or ventricle with conventional unipolar ablation techniques is very difficult to achieve. One of the main problems with this approach is the development of “collateral” damage i.e. damage to the neighboring structures such as the esophagus, phrenic nerves etc. Likely due to the technical difficulties, the success rates of catheter ablation for conditions such as persistent atrial fibrillation is poor. It has also been observed that ablation on muscle tissues has been associated with the development of inflammation and edema. Therefore, if the initial ablation attempts are unsuccessful in destroying the target tissue, it makes it less likely that subsequent applications from the same area will be successful (since, due to the inflammation, the target tissue will be further away from the ablating electrode).

Thus there is a need for improved ablation techniques and devices that achieve better necrosis without endangering the neighboring anatomical regions.

### BRIEF SUMMARY OF THE INVENTION

In one aspect, a dual catheter ablation system is provided to achieve effective necrosis of a target tissue. The system includes a catheter pair comprising an arterial catheter and a venous catheter. In one embodiment the system includes a first electrode in the arterial catheter and a second electrode in the venous catheter, where at least one of the first electrode or the second electrode is an ablating electrode. In another embodiment, only one of the arterial or venous catheter has an ablating electrode, while in another embodiment both catheters have ablating electrodes. Further, a first magnetic element of predefined polarity is placed at a space apart distance adjacent to the first electrode and a second magnetic element of opposite polarity with respect to the pre-defined polarity of the first magnetic element, is placed at a space apart distance adjacent to the second electrode. A protective sheath for enclosing the catheter pair, the first magnetic element and the second magnetic element may be provided. The dual catheter ablation system is configured to be placed inside an anatomical region such that a target tissue is in between the first electrode and the second electrode where both electrodes are present or between the first magnetic element and the second magnetic element.

### BRIEF DESCRIPTION OF THE SEVERAL VIEWS OF THE DRAWINGS

These and other features, aspects, and advantages of the present invention will become better understood when the following detailed description is read with reference to the accompanying drawings in which like reference numerals represent corresponding parts throughout the drawings, wherein:

FIG. 1 is an image for an abdominal region showing ganglionic plexi and nerve fibers anterior to the abdominal aorta and renal arteries;

FIG. 2 is a diagrammatic representation of an exemplary embodiment of the dual catheter ablation system of the invention where two sets of magnets or electromagnets are present. Magnets 18′ and 20′ are of opposite polarities and magnets (or electromagnets) 18 and 20 are of opposite polarities, and an ablation electrode is present in both the catheters;

FIG. 3 is a diagrammatic representation of another exemplary embodiment of the dual catheter ablation system of the invention where unlike FIG. 2, only one set of magnets (or electromagnets) of opposite polarities are present;

FIG. 4 is a diagrammatic representation of yet another embodiment of the dual catheter ablation system of the invention where an ablation electrode is present in only one catheter;

FIG. 5 is a diagrammatic representation of the “Namaste effect” implemented by use of the magnetic elements. The thumbs and the little fingers of the little fingers of the right and left hands represent magnetic or electromagnetic elements of opposite polarities. This figure illustrates that similar to the hands coming together during the salutation gesture, the catheters in the artery and vein will come together. As a result, the ablating electrode will now be adjacent to the target tissue;

FIG. 6 is another diagrammatic representation of the dual catheter system positioned in the artery and vein with the target tissue i.e. the nervous tissues are in between the two catheters;

FIG. 7 is a diagrammatic representation of the dual catheter system showing the magnetic pull of the catheters. As a result, the distance between the ablation element and the target tissue is greatly reduced. Therefore, the likelihood of a successful ablation is greatly enhanced;

FIG. 8 is a diagrammatic representation of the dual catheter system showing the ablation of the target tissue; During bipolar radiofrequency ablation, wavefronts of necrosis are seen to develop adjacent to both the ablation electrodes and progress towards each other, with the target tissue in between the wavefronts. Since the target tissue is ablated from two different directions, it is likely to be ablated successfully with application of lower power;

FIG. 9 is a diagrammatic representation showing progression of necrosis during bipolar radiofrequency ablation;

FIG. 10 is a graphical illustration showing the impedance changes in bipolar RF ablation;

FIG. 11 is a graphical illustration of impedance and time, and the corresponding progression of necrosis;

FIG. 12 illustrates the dependence of impedance on the number of living cells at any given time during RF ablation. For a given poser setting, once the impedance stops declining, it indicates that complete necrosis has been achieved;

FIG. 13 is a diagrammatic representation of a protective sheath for the catheters; and

FIG. 14 is a diagrammatic representation of two compartment balloon used with the catheters in the dual catheter system of the invention.

### DETAILED DESCRIPTION OF THE INVENTION

As used herein and in the claims, the singular forms “a,” “an,” and “the” include the plural reference unless the context clearly indicates otherwise.

With radiofrequency ablation, to overcome the disadvantages of the unipolar approach as described hereinabove, a bipolar approach where there are two catheters across the wall of the target tissue is considered more successful in creating transmural necrosis since an ablation lesion will develop adjacent to both catheter electrodes. In this technique, energy is delivered between two electrodes. This potentially results in a more focused delivery of energy which could also minimize lesion width and collateral tissue injury.

Unlike unipolar RF where a skin patch functions as an indifferent electrode (and therefore lesion develops adjacent only to the active electrode), here both electrodes function as active electrodes. Hence, lesions due to coagulation necrosis develop adjacent to both electrodes. A bipolar approach with lesion application across the wall is more likely to create transmural necrosis and achieve this with lower power. This approach where electromagnets are used to pull the two catheter with the intervening target tissue “sandwiched” in between (and thus the distance between the ablation element and the target tissue is greatly reduced) will also work with alternative energy sources such as delivery of heat, cryothermal energy, ultrasound, microwave and laser.

Another advantage of the technology (placing the tissue between two electrodes) is that it makes it possible to measure tissue impedance and conductance between the two electrodes and more accurately determine as to when necrosis has been achieved between the two electrodes. In contrast, there are no reliable techniques with unipolar RF ablation to detect the development of transmural necrosis.

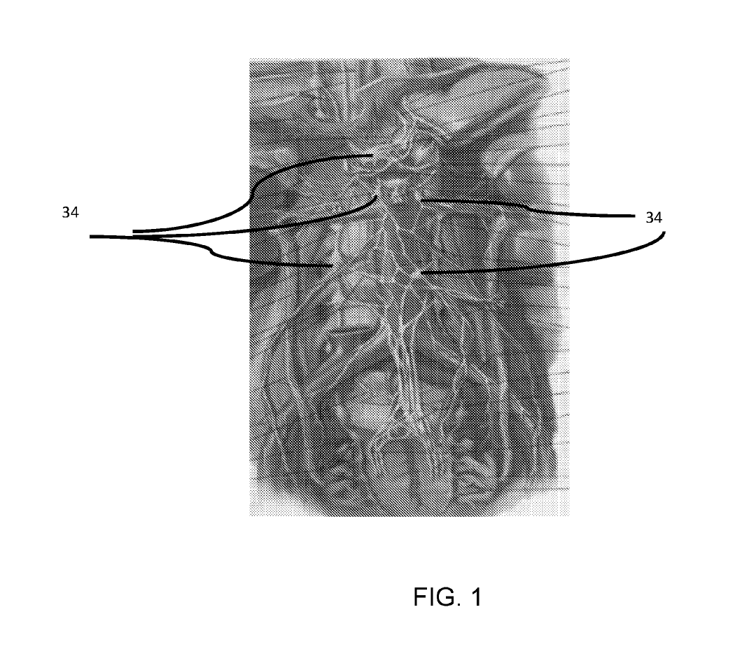

It would be known to those skilled in the art that in the abdominal region, the ganglionic plexi and nerve fibers tend to run on the anterior surface of the aorta and the renal vasculature and hence, along some stretches are between the vein and the arterial systems as shown in FIG. 1 and indicated by reference numeral 34. The method of the invention advantageously uses this geometrical feature of the heart for placing a novel dual catheter ablation system. The dual catheter ablation system described herein allows for a percutaneous ablation of the neurological tissues “in between” the arteries and veins and creates effective necrosis of these structures with the use of minimal power.

Thus the system and method of the invention enables the delivery of energy only along select geographic locations. Further, in a specific embodiment, the system and method of the invention enable ablation with a venous only rather than an arterial approach (the main purpose of delivering ablating energy from the vein is due to the fact that vein is larger than the artery, the venous wall is more flexible and is a “more forgiving structure” and therefore is safer for ablation procedure. Further, the system and method described herein enable implementation of effective bipolar ablation (or ablation performed simultaneously from the arterial and venous approaches). A further advantage of the system and method of the invention is use of electromagnetic or magnetic technology for effective placement of catheter system to access the target region. This approach will allow for a successful outcome with lower power use with fewer complications. The different exemplary non-limiting embodiments based on the above approach are described below in more detail.

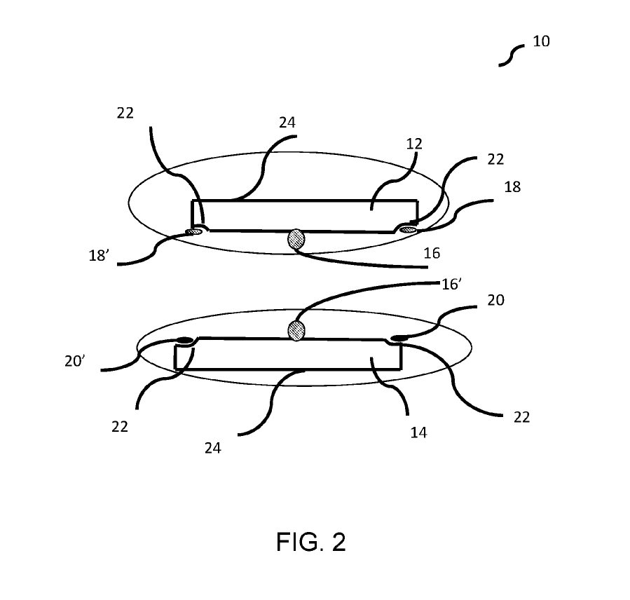

FIG. 2 is a diagrammatic representation of an exemplary embodiment of the dual catheter ablation system 10. It includes a catheter pair 12, 14 comprising an arterial catheter 12 with a first electrode 16 and a venous catheter 14 with a second electrode 16′. At least one of the first electrode or the second electrode is an ablating electrode. In a specific embodiment both electrodes are ablating electrodes. These ablating electrodes may be made of gold, platinum, silver or copper or other materials.

The dual catheter system further includes a first magnetic element 18 of predefined polarity placed at a space apart distance adjacent to the first electrode 16. Similarly, a second magnetic element 20 of opposite polarity with respect to the pre-defined polarity of the first magnetic element is placed at a space apart distance adjacent to the second electrode 16′. In the exemplary embodiment of FIG. 2, another magnetic element 18′ are shown and is positioned in a mirror image position of the element 18 with respect to the electrode 16. Similarly, a pair of opposite polarity magnetic elements 20′ is shown to be placed in a mirror image position of the element 20 with respect to the electrode 16′. In a specific implementations the magnetic elements 18 and 20 (and also 18′ and 20′) are electromagnets of opposite polarity. Alternately, in another example magnetic elements 18 and 18′ are electromagnets whereas magnetic elements 20 and 20′ are soft iron cores (or alternately, 18 and 18′ are soft iron cores and 20 and 20′ are electromagnets). To increase the electromagnetic field, a soft iron core may also be advanced through the coils of the electromagnet, number of turns can be adjusted or the current flow can be increased. Several other configurations of the magnetic elements may also be possible that achieve the purpose of attracting and aligning the two catheters in the region of interest.

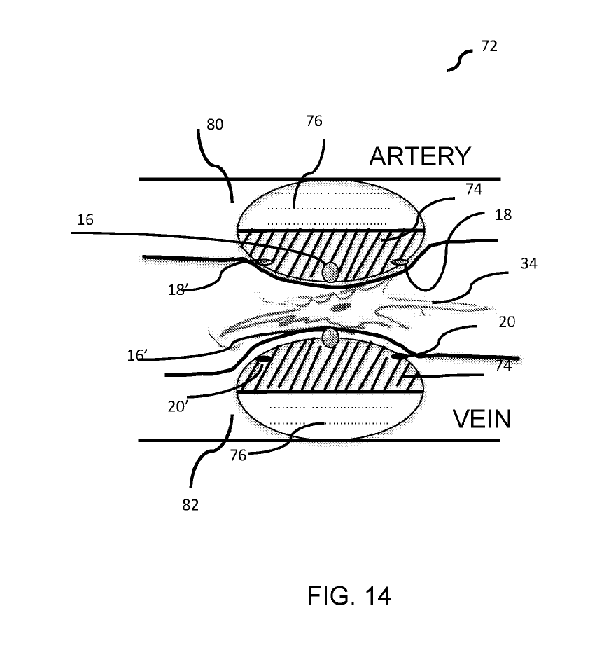

A protective sheath 24 is used for enclosing the catheter pair (shown in FIG. 14) for RF shielding (with polyamide, poly vinyl chloride or polyurethane or other materials) to prevent collateral damage to neighboring structures and to prevent leakage of RF energy into the blood stream. A protective ferrite sheath 22 is also used with the magnetic elements 18, 18′ and 20 and 20′.

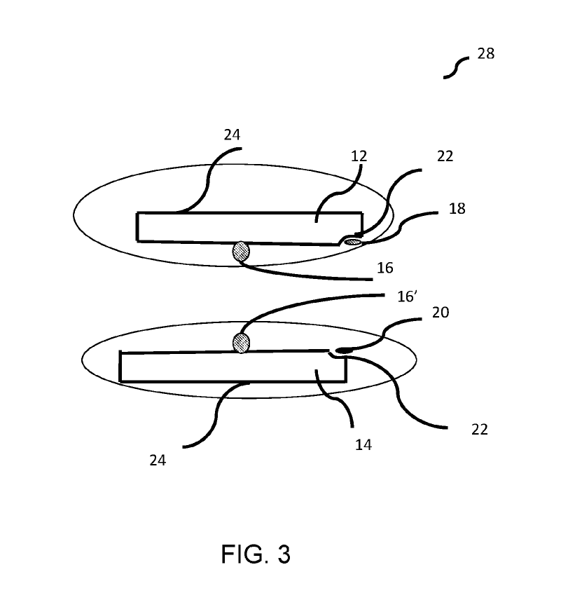

FIG. 3 describes another exemplary embodiment of the dual catheter system where only one magnetic element 18 is present on catheter 12 and only one magnetic element 20 is present on the catheter 14. The magnetic element 18 in one example is an electromagnet and the magnetic element 20 is a soft iron core. Alternately, the magnetic element 18 is a soft iron core and magnetic element 20 is an electromagnet in another example.

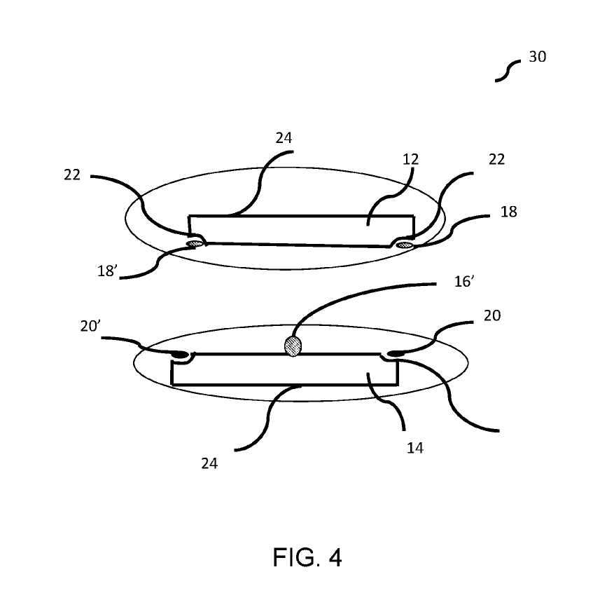

FIG. 4 describes another exemplary embodiment of the dual catheter system which is similar to FIG. 2 configuration except that only one ablating electrode 16′ is present on the venous catheter 14, the arterial catheter 12 lacks an ablating electrode. The sole function of the venous catheter here is to protect the neighboring structures from thermal injury. Hence in this configuration the venous catheter will deliver RF in the unipolar mode. The sole function of the arterial catheter in this configuration is to pull the venous catheter towards the target tissues i.e. the nerves and plexi that are present in between the two structures.

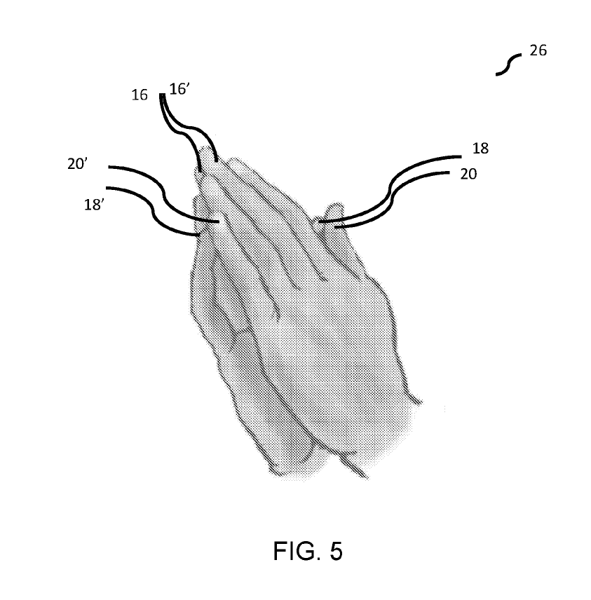

The electromagnets used in the different embodiments of the invention are configured to allow a Namaste effect, as shown in FIG. 5. The two thumbs of the hand represent the opposite poles of the magnet or electromagnet (18 and 20) placed in the artery and vein respectively. Similarly, the little fingers of the left and right and left hands represent the opposite poles of the electromagnets (18′ and 20′) in the artery and vein. The middle fingers represent the ablation electrodes 16 and 16′. This effect will ensure that the surface of the catheters that have the ablation electrodes in the artery and vein will face each other. Thus the electromagnets (with ferrite shielding) are used to induce the arterial and venous catheters to try and “stick” to each other across the walls and thus also improve contact of the electrode with the target tissues. Due to magnetic effect, movement of one catheter on one side of the target tissue will result in simultaneous movement of the other catheter on the other side of the tissue (similar to mirror image due to magnetic effect).

Different number of electromagnets may be used to produce the above effect, depending on factors including but not limited to the location and area of the target region, required strength of electromagnetic field, catheter thickness, length of electrode tip and other design parameters.

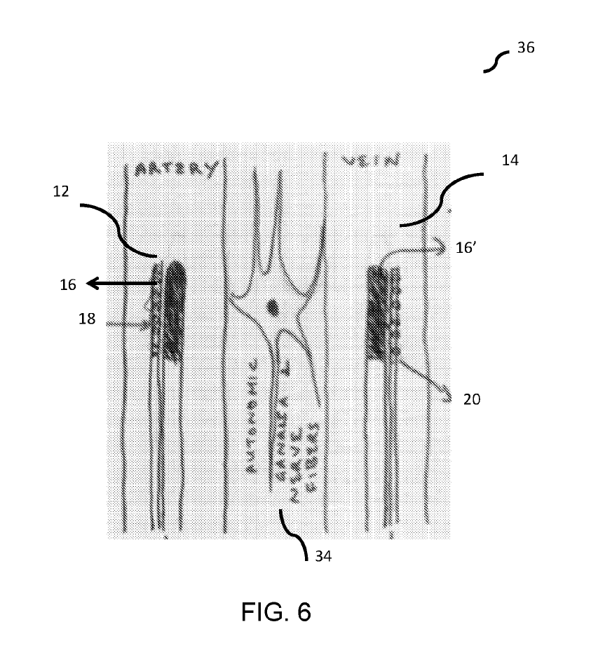

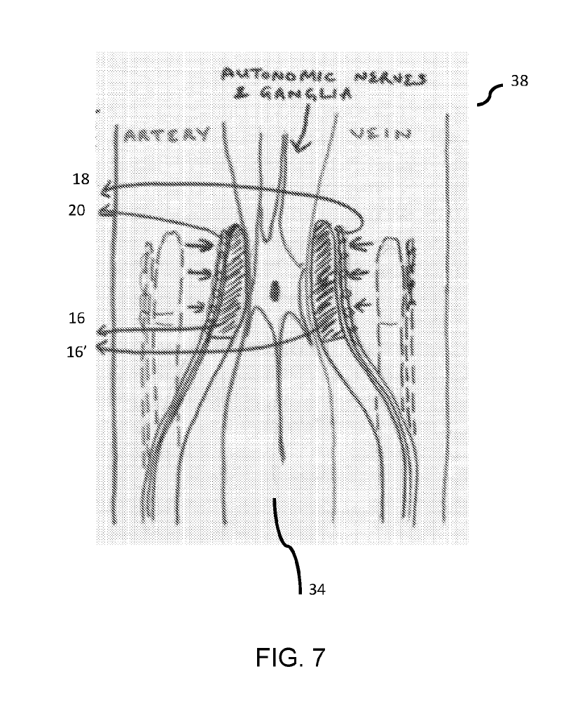

The dual catheter ablation system described herein is configured to be placed inside an anatomical region such that a target tissue 34 is in between the first electrode and the second electrode as shown in FIG. 6. In one specific implementation as shown in FIG. 6, the first catheter, the arterial catheter 12 having a first electrode 16 is placed in aorta and a second catheter 14, the venous catheter having a second electrode 16′ is placed in left renal vein and due to the magnetic elements 18 and 20, for example electromagnets of opposite polarities, the two catheters are aligned properly and pulled towards each other as shown in FIG. 7, thus dramatically increasing the ability of locally applied electromagnets to improve the contact or proximity of the ablation electrodes with the target tissues. RF current is delivered between the two catheters either as bipolar or unipolar RF as previously described. The other possible energy options include cryothermal energy, high energy focused ultrasound, microwave or laser.

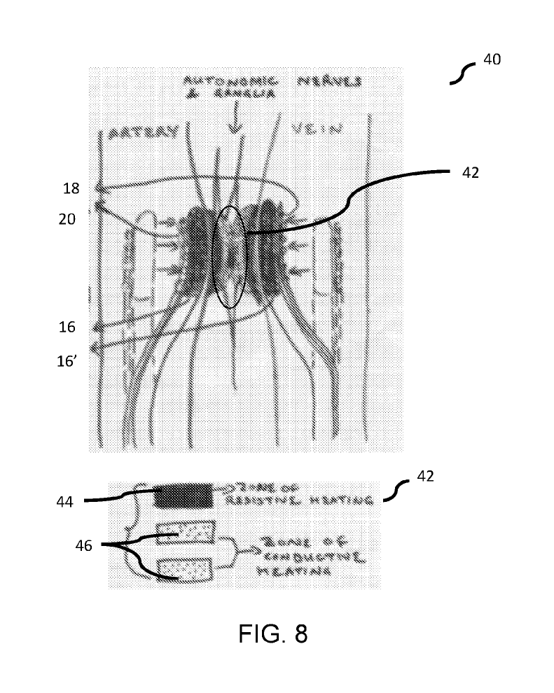

FIG. 8 illustrates the advantage of this novel technology and approach implemented using the dual catheter system. A wavefront of necrosis is seen to progress from two different directions (beginning from the two ablation electrodes), towards each other as shown by the region indicated by reference numeral 42 with the target tissue in between. With lesser power, satisfactory ablation of the target tissue is now achieved.

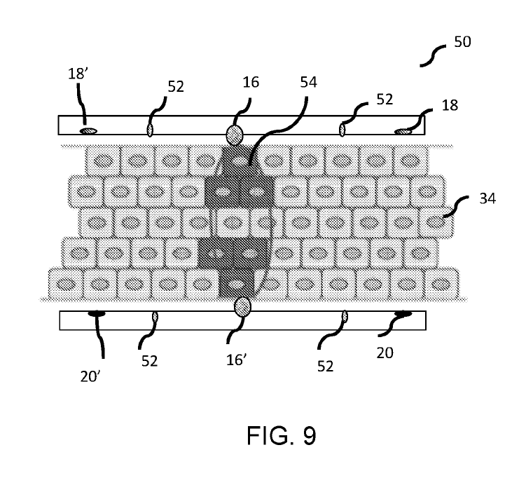

Another advantage of the technology (placing the tissue between 2 electrodes) also makes it possible to measure tissue impedance and conductance between the two electrodes and more accurately determine as to when necrosis has been achieved between the two electrodes. FIG. 9 illustrates a progression of necrosis in the region 54, dark cells indicating necrosis has been achieved. Additional electrodes 52 have been placed for impedance measurement in the region. When the impedance measurement is zero, it indicates that complete necrosis has been achieved and the RF generator can be stopped and ablation procedure is complete. Other techniques to determine completion of necrosis include optical spectroscopy and measurements such as capacitance, conductivity, phase angle measurements.

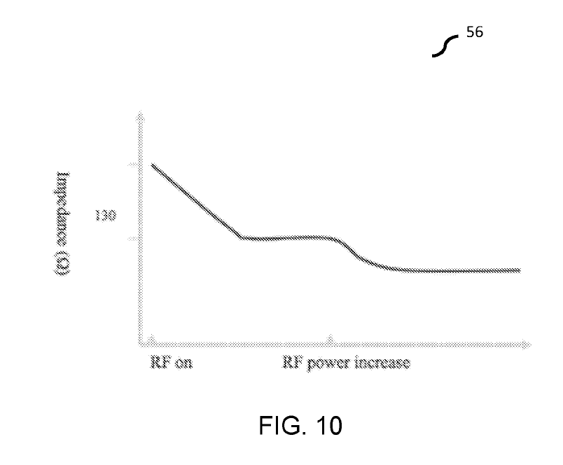

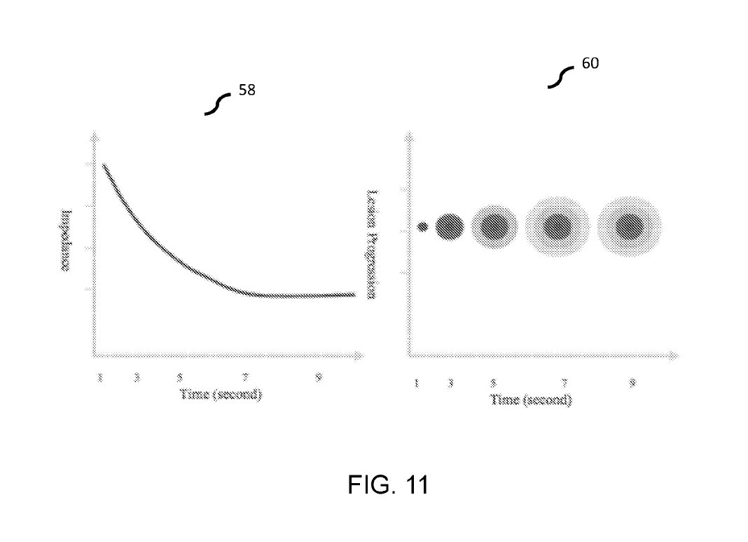

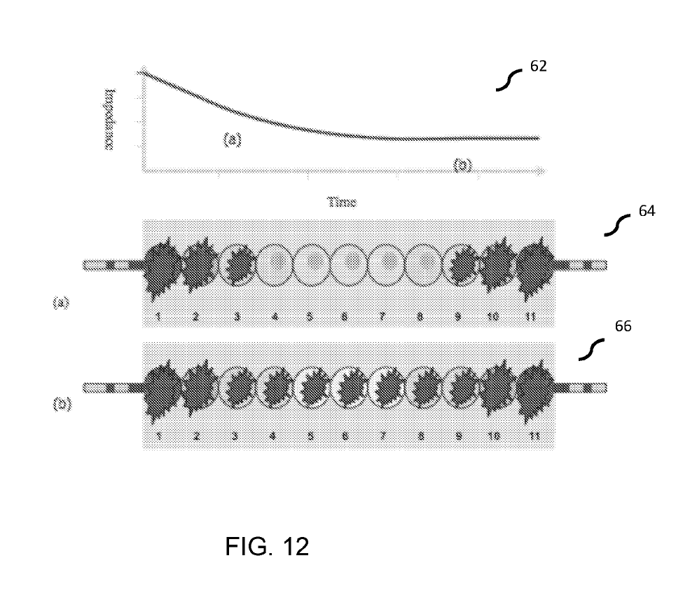

FIG. 10 is a graphical illustration 56 showing the impedance changes in bipolar RF ablation. Once RF energy is applied impedance drops quickly and is of greater magnitude in comparison with unipolar RF ablation. The impedance decreases and then plateaus. An increase in RF power triggers a further decrease in impedance and remains unchanged after reaching complete transmural necrosis. FIG. 11 is a graphical illustration 58 of impedance and time, and the corresponding progression of necrosis (lesion progression) in bipolar technique as indicated by numeral 60. As can be seen, the impedance decreases as size of lesion progressively enlarges and reaches plateau once lesion stops increasing in size. FIG. 12 illustrates the dependence of impedance on the number of living cells at any given time during RF ablation in the graph 62. As cells die, the impedance continues to drop as shown in region (a) shown diagrammatically by reference numeral 64. Once all the cells between the two electrodes are in necrotic state as in region (b), shown diagrammatically by reference numeral 66, impedance will no longer decline. Thus the dual ablation catheter system provides the unique advantage of ability to make measurements that indicate completion of necrosis that further improves the accuracy and efficacy of the ablation procedure.

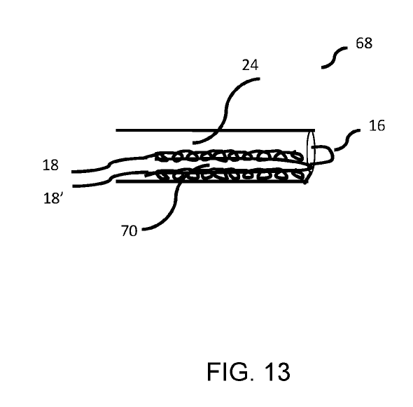

FIG. 13 is a diagrammatic representation of one of the catheter 68 of the dual catheter system that includes an electromagnetic sheath 24 is shown through which a standard ablation catheter 16 can be advanced through an opening 70. Electromagnets 18, 20 are also shown. In the exemplary implementation, a similar sheath will be placed on the other side (arterial or venous) as well. As mentioned earlier, RF shielding is provided for the ablation electrode system to prevent damage to neighboring structures. Further, shielding may also be added to the catheter to prevent leakage of RF or other energy into the blood stream.

In yet another specific implementation 72 balloon electrodes are used as shown in FIG. 14. A balloon 80 (or two balloons 80, 82) with a minimum of two compartments 74 and 76 is used with at least one of the catheters 12 or 14 (or with both catheters) and is accordingly positioned adjacent to at least one of the first electrode 16 and the second electrode 16′ that are used for ablating the nerve tissue 34. First compartment of the balloon 74 is designed to circulate hot fluid or cold fluid. The second compartment 76 is filled with a non conducting material or medium such as gas. The compartment that has the thermal energy delivering (74) ability faces towards the anterior wall of the aorta or the posterior wall of the IVC (inferior vena cava) or renal veins. The precise orientation of the different compartments of the balloons is facilitated by magnetic or electromagnetic elements.

In all the drawings the like numerals represent the like parts and have not been described again in subsequent drawings to avoid repeating for clarity in description.

The novel dual catheter ablation system and the ablation technique described herein provides several advantages as already described herein and allows specific targeted application of energy and limits the energy application during ablation to achieve renal sympathetic denervation and avoids delivering energy in a circumferential manner within a vessel that is done in prior art techniques.

While only certain features of the invention have been illustrated and described herein, many modifications and changes will occur to those skilled in the art. It is, therefore, to be understood that the appended claims are intended to cover all such modifications and changes as fall within the true spirit of the invention.

We claim:

1. A dual catheter ablation system comprising:
a catheter pair comprising an arterial catheter with a first electrode and a venous catheter with a second electrode, wherein at least one of the first electrode or the second electrode is an ablating electrode;
a first magnetic element of predefined polarity placed at a space apart distance adjacent to the first electrode;
a second magnetic element of opposite polarity with respect to the pre-defined polarity of the first magnetic element, placed at a space apart distance adjacent to the second electrode; and
a protective sheath for enclosing the catheter pair, the first magnetic element and the second magnetic element, wherein the dual catheter ablation system is configured to be placed inside an anatomical region such that a target tissue is in between the first electrode and the second electrode.

2. The dual catheter ablation system of claim 1 wherein at least one of the first magnetic element or the second magnetic element is an electromagnet.

3. The dual catheter ablation system of claim 1 wherein at least one of the first magnetic element or the second magnetic element is a soft iron core.

4. The dual catheter ablation system of claim 1 wherein the first magnetic element comprises a pair of electromagnets of the same predefined polarity, positioned in a mirror image location with respect to the first electrode.

5. The dual catheter ablation system of claim 1 wherein the second magnetic element comprises a pair of electromagnets of the same polarity, opposite to the predefined polarity, positioned in a mirror image location with respect to the second electrode.

6. The dual catheter ablation system of claim 1 wherein the protected sheath for the first magnetic element and the second magnetic element is a ferrite sheath.

7. The dual catheter ablation system of claim 1 wherein the protective sheath for the catheter pair is made of at least one of polyamide, poly vinyl chloride, and polyurethane and is configured to function as a radio frequency shield.

8. The dual catheter ablation system of claim 1 further comprising impedance measuring electrodes on each of the arterial catheter and the venous catheter to measure impedance during an ablation procedure.

9. The dual catheter ablation system of claim 1 wherein under operation the first magnetic element and the second magnetic element are attracted towards each other to bring the respective electrodes in close proximity.

10. The dual catheter ablation system of claim 1 further comprising a balloon positioned adjacent to at least one of the first electrode and the second electrode.

11. The dual catheter ablation system of claim 10 wherein the balloon is a two compartment balloon wherein a first compartment is filled with a non conducting medium and wherein the second compartment is filled with at least one of a hot fluid or a cold fluid, and wherein the second compartment faces the target tissue for transfer of thermal energy.

12. The dual catheter ablation system of claim 11 wherein the non conducting medium is a gaseous medium.

13. The dual catheter ablation system of claim 1 wherein the first electrode and the second electrode are activated using at least one of radio frequency current, cryoenergy, high energy focused ultrasound, microwave or laser.

14. The dual catheter ablation system of claim 13 wherein the radio frequency current is delivered in at least one of unipolar mode or bipolar mode.

15. A dual catheter ablation system comprising:
a catheter pair comprising an arterial catheter and a venous catheter with an ablating electrode;
a first magnetic element of predefined polarity placed in the arterial catheter;
a second magnetic element of opposite polarity with respect to the pre-defined polarity of the first magnetic element, placed at a space apart distance adjacent to the ablating electrode, wherein the first magnetic element and the second magnetic element are positioned to produce a clasping effect; and
a protective sheath for enclosing the catheter pair, the first magnetic element and the second magnetic element, wherein the dual catheter ablation system is configured to be placed inside an anatomical region such that a target tissue is in between the first magnetic element and the second magnetic element.
